(19) United States
(12) Patent Application Publication
(10) Pub. No.: US 2023/0348585 A1
(43) Pub. Date: Nov. 2, 2023

(54) METHODS FOR TREATING OR REDUCING THE SEVERITY OF A VIRAL INFECTION

(71) Applicants: St. Jude Children's Research Hospital, Inc., Memphis, TN (US); Washington University, St. Louis, MO (US)

(72) Inventors: Paul THOMAS, Memphis, TN (US); Jeremy Chase CRAWFORD, Memphis, TN (US); Emma Kaitlynn SLIGER, Memphis, TN (US); Aisha SOUQUETTE, Memphis, TN (US); Ali ELLEBEDY, St. Louis, MO (US); Philip MUDD, St. Louis, MO (US)

(21) Appl. No.: 17/923,029

(22) PCT Filed: May 5, 2021
(86) PCT No.: PCT/US2021/030789

§ 371 Date: Nov. 3, 2022

Related U.S. Application Data
(60) Provisional application No. 63/020,329, filed on May 5, 2020.

(51) Int. Cl. C07K 16/24; A61K 45/06

(52) U.S. Cl. CPC C07K 16/248; A61K 45/06

(57) ABSTRACT

Methods for treating or reducing the severity of a viral infection such as SARS-CoV-2 are provided, which include the administration of a cortisol antagonist preferably in combination with an IL-6 antagonist.

This application claims the benefit of priority from U.S. Provisional Application Ser. No. 63/020,329 filed May 5, 2020, the contents of which are incorporated herein by reference in their entirety. This invention was made with government support under Grant Number HHSN272201400006C awarded by the National Institutes of Health. The government has certain rights in the invention.

### BACKGROUND

Infection with severe acute respiratory syndrome coronavirus 2 (SARS-CoV-2) causes coronavirus disease 2019 (COVID-19). Acute respiratory failure occurs in a subset of COVID-19 patients. Understanding the etiology of respiratory failure in COVID-19 patients is critical for determining the best management strategies and pharmacologic targets for treatment. Current management of acute respiratory failure in COVID-19 includes optimized supportive care, primarily through oxygen administration and consideration of endotracheal intubation and mechanical ventilation in the appropriate context.

Cytokine storm syndrome (CSS) is increasingly proposed as underlying the etiology of respiratory failure in patients with COVID-19. This model suggests that respiratory failure is related to significant pro-inflammatory cytokine expression that leads to inflammatory cell recruitment and tissue damage in the lung. Most of the data supporting this hypothesis in COVID-19 comes from an early paper that observed high levels of the cytokines IL-2, IL-7, IL-10, G-CSF, IP-10, MCP-1, MIP-1 alpha and TNF-alpha in a small cohort of COVID-19 patients cared for in the intensive care unit (ICU). The level of these cytokines was increased in the ICU patients compared with a group of COVID-19 patients that did not require care in the ICU.³

There has been significant interest in modulating the systemic immune response in an effort to prevent or treat respiratory failure in patients with COVID-19. More than one hundred clinical trials are currently registered to evaluate the efficacy of inflammatory cytokine blocking medications or interventions such as cytokine filtration as potential treatments for respiratory failure in COVID-19 patients. A thorough understanding of the underlying inflammatory environment in COVID-19 patients is required to successfully interpret the findings of these studies.

### SUMMARY OF THE INVENTION

This invention provides methods for treating a viral infection or decreasing the severity of a viral infection by administering to a subject with such a viral infection an effective amount of a cortisol antagonist and optionally an IL-6 antagonist. In some aspect, the viral infection is a coronavirus infection such as SARS-CoV-2. A kit composed of a cortisol antagonist and an IL-6 antagonist is also provided.

### DETAILED DESCRIPTION OF THE INVENTION

To evaluate the etiology of respiratory failure in viral infections, a comparative investigation was conducted, wherein the inflammatory responses in a cohort of influenza patients with severe illness collected during 2019 and 2020 was compared to a cohort of COVID-19 patients. The analysis allowed for the characterization of the immune response in patients with severe COVID-19 specifically in the context of the more widely studied immune responses seen during influenza disease. This analysis identified a distinctive signature of immune regulation in patients with severe forms of COVID-19. This regulation consists of elevated levels of IL-6 in conjunction with other immune regulatory features consistent with persistent and elevated levels of glucocorticoid signaling. Accordingly, the present invention provides methods for treating, reducing severity and/or reducing clinical morbidity or mortality of virus infections, in particular coronavirus infections. The methods generally include administering a therapeutically effective amount of a cortisol antagonist, preferably in a combination with an IL-6 antagonist. In the description provided herein, a therapy for the treatment of a coronavirus infection, specifically SARS-CoV-2, is exemplified. However, the subject method is useful for treatment including prevention of any viral infection.

In accordance with the methods herein, a subject having, suspected of having, or at risk of having a viral infection is administered an effective amount of a cortisol antagonist and optionally an IL-6 antagonist to effect treatment or a reduction in the severity of the viral infection. In some embodiments, therapy is initiated after the appearance of clinical signs of a viral infection such as SARS-CoV-2, e.g., fever frequently exceeding 38° C. In some embodiments, therapy is administered prophylactically to the individual suspected of having a viral infection, e.g., a subject who is asymptomatic and not infected or yet infected, but has come into contact with an individual who has been diagnosed with a viral infection such as SARS-CoV-2; a subject who is asymptomatic and not yet infected but is diagnosed with a viral infection such as SARS-CoV-2; a subject who is expected to come into contact with individuals who have been diagnosed (e.g., health workers working in a facility where an individual who has been diagnosed with a viral infection such as SARS-CoV-2); or a subject who is traveling to a place where a relatively high onset is known. In some embodiments, the subject being treated has an upregulated level glucocorticoid signaling, e.g., as evidenced by gene expression analysis. Glucocorticoid signaling may be upregulated directly by the virus itself or by other factors including, e.g., physical and/or emotional stress, social isolation (Stafford, et al. (2013) Psychoneuroendocrinology 38(11):2737-45), diet (Tomiyama, et al. (2010) Psychosom. Med. 72(4):357-64), body mass index (Fraser, et al. (1999) Hypertension 33(6):1364-8), or disorders or dysfunction in a number of organs (e.g., adrenals, hypothalamus, pituitary).

Whether administered before or after the development of clinical signs of a viral infection, administration of a composition of the invention can reduce the risk that the subject will develop severe symptoms associated with a viral infection (e.g., SARS-CoV-2). In this respect, an effective amount of a cortisol antagonist is an amount that alone or in a combination therapy with an IL-6 antagonist reduces the risk or propensity for an individual to develop severe symptoms associated with a viral infection such as SARS-CoV-2 (e.g., hyperimmune response, clinical morbidity or mortality). For example, an effective amount is an amount that reduces the risk of developing severe symptoms by at least about 10%, at least about 20%, at least about 25%, at least about 30%, at least about 35%, at least about 40%, at least about 50%, at least about 60%, at least about 70%, at least about 80%, at least about 90% compared to the risk of developing severe symptoms associated with a viral infection such as SARS-CoV-2 in the absence of cortisol antagonist therapy. Whether severity decreases can be determined by measuring, e.g., cytokine levels or any symptom associated with a coronavirus infection including, for example, respiratory symptoms (e.g., cough, easy or difficult breathing), and the like. Treatment or a reduction in severity can shorten the time the subject is sick, decrease reliance on a ventilator, decrease the time the subject is hospitalized, etc.

The term “cortisol antagonist” refers to any compound or agent which reduces production of cortisol or circulating levels of biologically active cortisol or which limits the biological effects of cortisol by inhibiting cortisol (glucocorticoid) receptors competitively or non-competitively, or in any other way which interferes with the regulation of cortisol synthesis along the so-called hypothalamic-pituitary adrenal gland axis. Thus, a “cortisol antagonist” may broadly be regarded as any compound or agent which antagonizes or inhibits or reduces or prevents) cortisol activity.

A number of agents are known to suppress glucocorticoid production or inhibit their receptor binding in humans including, e.g., sodium valporate (Aggernaes, et al. (1988) Acta Psychiatr. Scand. 22:170-174); Enkephalins and their synthetic analogues (Stubbs, et al. (1978) Lancet 11:1225-1227); Clonidine (Slowinska-Srzednicka, et al. (1988) Eur. J. Clin. Pharmacol. 35:115-121); Oxytocin (Legros, et al. (1987) Endocrinologica 114:345-349) and Mifepristone, known as RU 486 or RU 38486. Any of the above agents or any of the large number of cortisol synthesis inhibitors known in the art, e.g., those containing an azole group such as econazole, ketoconazole, levoketoconazole, fluconazole, itraconazole and miconazole and their derivatives, may be used as cortisol antagonists according to the present invention. Other agents known to have effects on cortisol secretion and/or activity include aminoglutethimide (sold under the tradename Elipten®), metyrapone (sold under the tradename Metopirone®), etomidate, trilostane, mitotane (sold under the tradename Lysodren®), pasireotide and trilostan. Phenyltoin (dilantin, diphenylhydantoin, DPH), procaine, vitamin C, salicylates including aspirin, cimetidine and lidocaine are further pharmaceuticals for which a cortisol antagonistic activity has been observed. Also, other imidazole derivatives than ketoconazole and its derivatives are included in the invention. Furthermore, certain phosphatidylcholines and serines are currently promoted as cortisol synthesis inhibitors for the treatment of increased cortisol secretion induced by the stress caused by too much exercise. Further cortisol antagonists, particularly melengesterol acetate and its derivatives, are described in U.S. Pat. No. 5,252,564.

Preferred cortisol antagonists include those compounds which inhibit the synthesis of cortisol, either by reducing the production of cortisol in any form or which cause the production of a modified form of cortisol which is less biologically active than native, naturally occurring cortisol. Preferably, cortisol synthesis inhibitors will act on the cortisol synthetic pathway in a way which does not significantly affect the normal production of the other steroid hormones. In certain embodiments, the cortisol antagonist maintains normal cortisol levels or returns elevated cortisol levels to a normal level. The term “normal cortisol level” refers to the average level of cortisol as determined by measurements of samples (e.g., serum samples) obtained from multiple normal subjects.

In certain aspects, the cortisol antagonist is administered in combination with an IL-6 antagonist. As used herein, the term “IL-6 antagonists” refers to a substance which inhibits or neutralizes the angiogenic activity of IL-6. Such antagonists accomplish this effect in a variety of ways. One class of IL-6 antagonists will bind to IL-6 protein with sufficient affinity and specificity to neutralize the angiogenic effect of IL-6. Included in this class of molecules are antibodies and antibody fragments (such as for example, F(ab) or F(ab′)2 molecules). Another class of IL-6 antagonists are fragments of IL-6 protein, muteins or small organic molecules, i.e., peptidomimetics, that will bind to IL-6, thereby inhibiting the angiogenic activity of IL-6. The IL-6 antagonist may be of any of these classes as long as it is a substance that inhibits IL-6 angiogenic activity. IL-6 antagonists include IL-6 antibody, IL-6R antibody, an anti-gp130 antibody or antagonist, modified IL-6 such as those disclosed in U.S. Pat. No. 5,723,120, antisense IL-6R and partial peptides of IL-6 or IL-6R.

Any of the anti-IL-6 antibodies known it the art may be employed in the method of the present invention. Murine monoclonal antibodies to IL-6 are known as in, for example, U.S. Pat. No. 5,618,700 or the antibody known as B-E8 (Diaclone, France) or the antibody referred to as CLB-6/8 capable of inhibiting receptor signaling (Brakenhoff, et al. (1990) J. Immunol. 145:561) may be used. Examples of antibodies that bind to the IL-6 receptors include MR16-1 antibody (Tamura, et al. (1993) Proc. Natl. Acad. Sci. USA 90:11924-11928), PM-1 antibody (Hirata, et al. (1989) J. Immunol. 143:2900-2906), AUK12-20 antibody, AUK64-7 and A0K146-15 antibody (WO 92/19759).

To avoid immune response to the antibody which causes adverse effects as well as eliminating the therapeutic action of the antibody, it is desirable to administer a human or close to human antibody scaffold. U.S. Pat. No. 5,856,135 discloses reshaped antibodies to human IL-6 derived from a mouse monoclonal antibody SK2 in which the complementary determining regions (CDR's) from the variable region of the mouse antibody SK2 are transplanted into the variable region of a human antibody and joined to the constant region of a human antibody. A chimerized form of the murine IL-6 monoclonal of the CLB-6/8 murine antibody called cCLB8 has been constructed (Centocor, Leiden, The Netherlands) and given to multiple myeloma patients (Van Zaanen, et al. (1996) J. Clin. Invest. 98:1441-1448). Other process for humanizing of primatizing antibodies raised in non-human species are also suitable for constructing antibodies of the present invention providing the product antibody retains its ability to block IL-6 from signaling in the target cell through interaction with its cognate receptor or receptor complex.

Other agents affecting a decrease in IL-6, such as the IL-6 receptor antagonist Sant7 (Tassone, et al. (2002) Int. J. Oncol. 21:867-873) may also be employed.

The cortisol antagonist and optional IL-6 antagonist may be administered individually or co-formulated to treat a viral infection and/or decrease the severity of a viral infection. In general, the antagonists described herein will be formulated as a pharmaceutical composition in admixture with a pharmaceutically acceptable excipient according to known methods. See, e.g., Gennaro (2000) Remington: The Science and Practice of Pharmacy, 20th edition, Lippincott, Williams & Wilkins; Pharmaceutical Dosage Forms and Drug. Delivery Systems (1999) Ansel et al., 7th edition, Lippincott, Williams & Wilkins; and Handbook of Pharmaceutical Excipients (2000) A H Kibbe et al., 3rd edition, Amer. Pharmaceutical Assoc.

By “pharmaceutically acceptable” it is meant that the ingredients must be compatible with other ingredients of the composition as well as physiologically acceptable to the recipient. Pharmaceutically acceptable excipients such as vehicles, adjuvants, carriers or diluents are generally readily available. In addition, pharmaceutically acceptable auxiliary substances such as pH adjusting and buffering agents, isotonic agents, stabilizers, wetting agents and the like are generally readily available.

The pharmaceutical compositions may be formulated according to any of the conventional methods known in the art and widely described in the literature. Thus, the active ingredient may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions sterile packaged powders, and the like. In this respect, the pharmaceutical composition can be administered in various ways, for example, oral, buccal, rectal, parenteral, intraperitoneal, intradermal, subcutaneous, intramuscular, transdermal, intranasal, intrapulmonary, intratracheal, etc.

Examples of suitable carriers, excipients, and diluents are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methylhydroxyoenzoates, propyl hydroxybenzoates, talc, magnesium stearate, mineral oil or fatty substances such as hard fat or suitable mixtures thereof. The compositions may additionally include lubricating agents, wetting agents, emulsifying agents, suspending agents, preserving agents, sweetening agents, flavoring agents, and the like. The compositions of the invention may be formulated so as to provide quick, sustained or delayed release of the active ingredient after administration to the patient by employing procedures well known in the art.

Suitable doses will vary from patient to patient and can be determined by the physician in accordance with the weight, age and sex of the patient and the viral infection and also the particular antagonist selected. A typical total daily dose may be in the range of 0.5 mg to 50 mg per kilogram body weight of a cortisol antagonist, which may be administered as a single dose or in several smaller doses during the day. By way of illustration, a typical pharmaceutical formulation for oral administration of mifepristone is in a daily amount of between about 0.5 to about 20 mg per 15 kilogram of body weight per day.

Similarly, an effective dose of an IL-6 antagonist may be in the range of 0.01 mg to 100 mg per kilogram of body weight per administration. Alternatively, a dose of 1 to 1000 mg, and preferably 5 to 50 mg, can be selected per subject.

Subjects or patients suitable for treatment with a pharmaceutical composition or formulation may be identified by well-established indicators of the risk of developing the disease or by well-established characteristics of the disease present. For example, indicators of viral infection include fever, dry cough, shortness of breath (shortness of breath), headache, hypoxemia (low blood oxygen levels), lymphopenia (reduced lymphocyte count), and slightly elevated aminotransferase levels (liver damage). In certain embodiments, subjects suitable for treatment exhibit the phenotype of high IL-6 expression, low IFN signaling, and profound cytopenias.

Viral infections that can be treated with the therapy described herein include infections caused by or because of an arenavirus, coronavirus, filovirus, orthomyxovirus, paramyxovirus, or retrovirus family of viruses. In certain embodiments, the viral infection is caused by or because of a virus selected from the group of Lassa Virus, Lymphocytic Choriomeningitis Virus (LCMV), Junin Virus, Machupo Virus, Guanarito Virus, Sabia Virus, Severe Acute Respiratory Syndrome (SARS) Virus, Murine Hepatitis Virus (MHV), Human Coronavirus, Bovine Coronavirus, Canine Coronavirus, Feline Infectious Peritonitis Virus, Ebola Virus, Marburg Virus, Influenza A Virus, Influenza B Virus, Influenza C Virus, Measles Virus, Mumps Virus, Canine Distemper Virus, Newcastle Disease Virus, Human Immunodeficiency Virus 1 (HIV-1), Human Immunodeficiency Virus 2 (HIV-2), Human T-cell Lymphotrophic Virus 1 (HTLV-1), Human T-cell Lymphotrophic Virus 2 (HTLV-2), Human Intracisternal A-type Particle 1 (HIAP-1), and Human Intracisternal A-type Particle 2 (HIAP-2). In certain embodiments, the subject being treated has been diagnosed with a coronavirus, in particular an α-type human coronaviruses (HCoVs) such as HCoV-229E and HCoV-NL-63; β-type HCoV-HK01, SARS-CoV, MERS-CoV, and HCoV-0C43; or 2019-nCoV (i.e., SARS-CoV-2).

The present invention also provides a kit for carrying out the methods of this present invention. The kit of the invention includes at least one cortisol antagonist in a therapeutically effective amount and at least one IL-6 antagonist in a therapeutically effective amount. The kit may provide a single dose or multiple doses of cortisol antagonist(s) and IL-6 antagonist(s), wherein said antagonists may be provided individually or in a co-formulation. The kit may take the form of a blister package; a lidded blister, a blister card or packet; a clamshell; an intravenous (IV) package, IV packette or IV container, a tray or a shrink wrap comprising the antagonists and instructions for use of the composition for treating or reducing the severity of a viral infection such as SARS-CoV-2.

The following non-limiting examples are provided to further illustrate the present invention.

### EXAMPLE 1: MATERIALS AND METHODS

Study Design. A prospective observational cohort study was conducted for subjects with viral respiratory illness symptoms who presented to Barnes Jewish Hospital, St. Louis Children's Hospital, Missouri Baptist Medical Center or affiliated Barnes Jewish Hospital testing sites located in Saint Louis, MO. Inclusion criteria required that subjects were symptomatic and had a physician-ordered SARS-CoV-2 test performed in the course of their normal clinical care. Some subjects were enrolled before the return of the SARS-CoV-2 test result. Enrolled subjects who tested negative for SARS-CoV-2 were not included. This analysis includes the first subjects enrolled in the study. The first 79 SARS-CoV-2⁺ subjects were the primary cohort and the next 89 enrolled SARS-CoV-2⁺ subjects were the validation cohort. All samples were collected at the time of enrollment, and there was a median interval between hospital admission and enrollment of 1 day (which corresponded to the median turn-around time for the SARS-CoV-2 clinical reverse transcription PCR test). Patient-reported duration of illness and other clinically relevant medical information was collected at the time of enrollment from the subject, their legally authorized representative, or the medical record. The vast majority of samples from patients testing positive for SARS-CoV-2 were collected immediately after the treatment team learned that the subjects were positive, and therefore, most patients will not have received any specific treatment before sample collected. Less information was obtained about whether treatment had begun before sample collection for the validation cohort. The primary and validation cohorts were considered separately herein. The portions of the study relevant to each institution were reviewed and approved by the Washington University in Saint Louis Institutional Review Board and the Missouri Baptist Medical Center Institutional Review Board. The study complied with the ethical standards of the Helsinki Declaration.

Findings from healthy control subjects and influenza-infected subjects enrolled in separate, ongoing studies are also reported. Control subjects had not experienced symptoms of a viral respiratory illness at the time of sample collection or within the previous 90 days, and samples were all collected before October of 2019. Influenza subjects were enrolled in the ongoing EDFLU study (Turner, et al. (2020) J. Infect. Dis. 222:1235-44). All influenza subjects were sampled in 2019 and 2020. Most influenza subjects were enrolled during the course of the 2019 to 2020 influenza season, immediately before the spread of COVID-19 disease in the Saint Louis region. The last included influenza subject was enrolled and sampled on 2 Mar. 2020. The first case of COVID-19 was reported in Saint Louis on 8 Mar. 2020 in a returning traveler. The control and influenza studies were independently approved by the Washington University Institutional Review Board.

Multi-Parameter Flow Cytometry. Absolute counts of CD45⁺ cells in whole blood were determined at the time of blood collection on fresh samples by flow cytometry with Precision Count Beads (BioLegend). Peripheral blood mononuclear cells (PBMCs) were prepared with copolymers of sucrose and epichlorohydrin sold under the tradename FICOLL®. PBMCs were analyzed using a panel of antibodies directed against the following antigens: CD8 BV421 (clone RPA-T8), CD20 Pacific Blue (clone 2H7), CD16 BV570 (clone 3G8), HLA-DR BV605 (clone L243), immunoglobulin D (IgD) SuperBright 702 (clone IA6-2), CD19 BV750 (clone HIB19), CD45 (labeled with a fluorescent dye sold under the tradename ALEXA FLUOR® 532, clone HI30), CD71 PE (clone CY1G4), CD38 PE-Cy7 (clone HIT2), CD14 APC (clone M5E2), CD4 Spark 685 (clone SK3), and CD3 (labeled with a fluorescent dye sold under the tradename ALEXA FLUOR® 700, clone UCHT1. PBMC samples of 0.5-2×10⁶ cells were stained with a master-mix containing pre-titrated concentrations of the antibodies, along with BD Brilliant™ Buffer (BD Biosciences) and Zombie NIR™ Fixable Viability Marker (BioLegend) to differentiate live and dead cells. Samples were run on a Cytek Aurora spectral flow cytometer using SpectroFlo software (Cytek) and unmixed before final analysis was completed using FlowJo software (BD Biosciences).

Cytokine Quantification. Plasma obtained from subjects was frozen at −80° C. and subsequently analyzed using a human magnetic cytokine panel providing parallel measurement of 35 cytokines (Thermo Fisher Scientific). The assay was performed according to the manufacturer's instructions with each subject sample performed in duplicate and then analyzed on a Luminex™ FLEXMAP 3D™ instrument.

Single-Cell RNA Sequencing. PBMCs were suspended at 1000 cells/μL and approximately 17,400 cells were input to a 10× Genomics Chromium instrument. Aside from healthy control sample ZW-WU321, each sample was used for two independent reactions, with all first reactions processed on one chip and second reactions processed on a second chip. Single-cell gene expression libraries were prepared using 5′ (V2) kits and sequenced on the Illumina NovaSeq 6000 platform at 151×151 base pair. Individual libraries were processed using CellRanger (v3.1.0; 10× Genomics) with the accompanying human reference (GRCh38-3.0.0), which was modified to include the influenza A, influenza B, and COVID-19 (NC_04551.2.2) genomes. Processed libraries were subsequently aggregated using CellRanger, randomly subsampling mapped reads to equalize sequencing depth across cells. Filtered aggregation matrices were subsequently analyzed using Seurat (v3.1.4; Stuart, et al. (2019) Cell 177:1888-902.e21), excluding cells from downstream analyses that exhibited extremes in the total number of transcripts expressed, the total number of genes expressed, and mitochondrial gene expression. For each cell, cell cycle phase was inferred using established markers (Tirosh, et al (2016) Science 352:189-96) and module scores were incorporated from a number of external gene sets in the same manner.

After filtering, putative cell subsets shared across conditions were identified by detecting integration anchors among the samples, effectively minimizing condition-associated differences. The top 2000 variable genes were identified for each library using the “vst” method, and integration anchors were obtained using canonical correlation analysis (CCA). Data were integrated using 50 CCA dimensions and scaled to regress out the effects of total transcript count, percent of mitochondrial gene expression, and module scores associated with cell phase. Principal components (PC) were calculated and assessed for statistical significance using random permutation. The first 45 PCs (P<0.01) were used to identify transcriptional clusters and for t-distributed stochastic neighbor embedding and UMAP (uniform manifold approximation and projection) dimensionality reduction. After identifying clusters on the basis of transcriptional similarities across cells from all three conditions (i.e., the “integrated” analysis), pairwise differential gene expression analysis was performed between conditions using Wilcoxon rank sum tests as implemented in Seurat, with default parameters. An additional UMAP projection was also generated using the top 2000 variable genes across the entire dataset (excluding T cell receptor and immunoglobulin (IG) genes, which are known to map poorly) irrespective of the CCA but again using significant PCs. This allowed for visualization of cells in a manner that did not obscure transcriptional differences owed to sample or condition but with previously identified cell subsets and transcriptional clusters from the integration analysis overlaid. Identified subsets and clusters were subsequently analyzed for explicit differences in gene pathway enrichment between cells from COVID-19-infected and influenza-infected patients, COVID-19-infected and healthy participants, and influenza-infected and healthy participants. Gene expression differences between conditions were ranked for individual subsets and transcriptional clusters by calculating differential expression under a generalized linear hurdle model (Finak, et al. (2015) Genome Biol. 16:278). To generate gene ranks, gene-specific average log fold changes were multiplied by the absolute difference in the proportions of cells expressing the gene (1×10⁻⁴ as a lower boundary) and the inverse of the Bonferroni-corrected P values, which were resealed from 1×10⁻⁷ to 1 to institute reasonable bounds in the ranking. This approach has been implemented previously (Cortez, et al. (2020) Nat. Commun. 11:2097) to synthesize information about average expression differences, the fraction of cells expressing the gene at all, and statistical assessments of significance. These ranks were used as inputs for gene set enrichment analysis (Subramanian, et al. (2005) Proc. Natl. Acad. Sci. USA 102:15545-50) using GSEA Preranked with a classic enrichment statistic and chip-based gene collapsing based on the Human_Symbol_with_Remapping_MSigDB.v.7.0 chip (Liberzon, et al. (2011) Bioinformatics 27:1739-40). Gene sets were considered significantly enriched if they resulted in a nominal P value of <0.05 and a q value of <0.20.

Cohort Comparisons. Statistical analyses of primary cohort demographics included pairwise comparisons between COVID-19 and healthy groups for age, sex, and ethnicity using a multinomial logistic regression, with the variation from the influenza group included in the model. Results were reported as odds ratio and (when significant) corresponding P value. Comparisons between COVID-19 and influenza groups were performed using a multivariate logistic regression between the COVID-19 and influenza groups only, as the clinical variables were irrelevant to healthy controls. The “immunocompromised” comorbidity was not included in the primary cohort logistic regression because it perfectly segregated across groups. The COVID-19 validation cohort was compared to the COVID-19, healthy, and influenza groups from the primary cohort again using multinomial logistic regression, with comparisons between validation and healthy groups limited to demographic variables. Multinomial regressions were modeled using the R “nnet” package (v7.3.14; Venables & Ripley (2002) Modern Applied Statistics with S, Ed. 4, Springer), and multivariate logistic regressions were modeled using the “glm” function in R. P values were adjusted for multiple testing by controlling the Benjamini-Hochberg false discovery rate approach.

Flow Cytometry. Flow cytometry measures were compared across healthy, influenza, and SARS-CoV-2 subjects using multivariate linear regression, with login subset percentages, counts, or mean fluorescence intensities modeled as a function of condition (COVID-19-infected, influenza-infected, and healthy control) with sex, age, ethnicity, and all comorbidities included as covariates among comparisons to healthy controls, and all of those covariates, as well as the number of days since symptom onset at study enrollment included in comparisons between influenza-infected and COVID-19-infected patients. The “emmeans” package in R was used to assess pairwise differences in estimated marginal means between conditions or severity, and Tukey's method was used to adjust for multiple comparisons. In the HLA-DR expression analysis, there were four negative mean fluorescence intensity observations, and these were replaced with a value of 1 before analysis.

Cytokines. PCA was conducted on samples without missing data points using the “prcomp” function in R and visualized using the “factoextra” package. Cytokine concentrations were otherwise compared across healthy, influenza, and SARS-CoV-2 subjects using multivariate linear regression, with login concentration modeled as a function of condition (COVID-19-infected, influenza-infected, and healthy control) with sex, age, ethnicity, and all comorbidities included as covariates among comparisons to healthy controls, and all of those covariates, as well as the number of days since symptom onset at study enrollment, included in comparisons between influenza-infected and COVID-19-infected patients. The “emmeans” package in R was used to assess pairwise differences in estimated marginal means between conditions, and Tukey's method was used to adjust for multiple comparisons. Data points from CSS samples were not included in the statistical analyses so as to prevent skewing the results. Data points from T_{H}22 samples were included in the validation cohort COVID-19 samples for analysis even when visualized separately.

Cytokine-cytokine co-correlations were investigated using CytoMod (Cohen, et al. (2019) Front. Immunol. 10:1338) using absolute cytokine concentrations of COVID-19 samples across both the primary and validation cohorts. For these correlations, values below the lower limit of detection were set to the lower limit of detection, and values above the upper limit of detection were set to the upper limit of detection. Up to k=12 modules were tested and the change in gap statistic was used to identify the optimal k.

Logistic regressions for severity were carried out within R and included sex, age, ethnicity, the number of days since symptom onset, cohort, and all comorbidities as covariates. P values were adjusted for multiple testing by controlling the false discovery rate as described above. Forest plots were generated using the “forestplot” package in R.

### EXAMPLE 2: DEMOGRAPHIC AND CLINICAL CHARACTERISTICS

A total of 79 symptomatic subjects who tested positive for SARS-CoV-2 RNA using a Food and Drug Administration-approved clinical polymerase chain reaction (PCR) test were enrolled in the initial (primary) cohort. The comparison group was composed of 26 symptomatic seasonal influenza subjects recruited during the period of 15 months immediately preceding the outbreak of COVID-19 in the Saint Louis region, all of whom tested positive for influenza A or B via a clinical PCR test obtained during their clinical care. COVID-19 subjects were, on average, 19 years older than influenza subjects and 29 years older than control subjects (Table 1). A greater number of COVID-19 subjects required hospitalization, ICU admission, and mechanical ventilation than influenza subjects, but this was not significantly different after controlling for demographic factors and other clinical characteristics (Table 2). Twenty-seven percent of the COVID-19 subjects died during their hospitalization compared with 8% of influenza subjects enrolled. Many subjects in both influenza and COVID-19 groups exhibited comorbidities that increased their risk for severe disease, including diabetes and chronic lung disease; however, there were no significant differences between the COVID-19 and influenza subjects in any analyzed comorbidity (Table 2). Both the COVID-19 and influenza cohorts included subjects with moderate disease, as defined by individuals with symptomatic illness requiring evaluation in the hospital, and severe disease, as defined by individuals requiring mechanical ventilation for acute respiratory failure or who ultimately died due to their illness.

TABLE 1

| | SARS- | Healthy | | COVID-19- | COVID-19- |
|---|---|---|---|---|---|
| | CoV-2 | Control | Influenza | Healthy | Influenza |
| | (n = 79) | (n = 16) | (n = 26) | Comparison | Comparison |
| Demographics | | | | | |
| Means ± SD | 61 ± 15 | 32 ± 7 | 42 ± 17 | P < 0.001, | P = 0.007, |
| (range) | (25-89) | (22-49) | (18-89) | OR = 0.85 | OR = 0.93 |
| age, in | | | | | |
| years | | | | | |
| Female | 44% | 50% | 58% | P = 1, | P = 1, |
| | (35/44) | (8/8) | (15/26) | N.S. | N.S. |
| Ethnicity | | | | | |
| African | 80% | 44% | 65% | — | — |
| American | (63/79) | (7/16) | (17/26) | | |
| White | 18% | 56% | 27% | P < 0.05, | P = 0.718, |
| | (14/79) | (9/16) | (7/26) | OR = 9.59 | N.S. |
| Other | <3% | 0% | 8% | — | P = 1, N.S. |
| | (2/79) | (0/16) | (2/26) | | |

N.S., not significant.

TABLE 2

| | SARS- | | COVID-19- |
|---|---|---|---|
| | CoV-2 | Influenza | Influenza |
| | (n = 79) | (n = 26) | Comparison |
| Clinical Characteristics | | | |
| Mean (IQR) symptom duration | 6.4 | 4.1 | p = 0.229, |
| at study enrollment, in days | (3-9) | (2-7) | N.S. |
| Hospital admission | 90% | 58% | P = 0.229, |
| | (71/79) | (15/26) | N.S. |
| ICU admission | 56% | 35% | P = 0.285, |
| | (44/79) | (9/26) | N.S. |
| Intubation and mechanical | 44% | 27% | P = 0.285, |
| ventilation | (35/79) | (7/26) | N.S. |
| In-hospital death | 30% | 8% | P = 0.234, |
| | (24/79) | (2/26) | N.S. |
| Comorbidities | | | |
| Immunocompromised | 6% | 0% | P = 0.33, |
| | (5/79) | (0/26) | N.S. |
| Chronic lung disease | 34% | 42% | P = 0.682, |
| | (27/79) | (11/26) | N.S. |
| Chronic heart failure | 13% | 23% | P = 0.101, |
| | (10/79) | (6/26) | N.S. |
| End-stage renal failure | 5% | 8% | P = 0.582, |
| | (4/79) | (2/26) | N.S. |
| Diabetes mellitus | 43% | 27% | p = 0.628, |
| | (34/79) | (7/26) | N.S. |
| Active cancer | 6% | 8% | P = 0.234, |
| | (5/79) | (2/26) | N.S. |

N.S., not significant.

### EXAMPLE 3: EVALUATION OF CIRCULATING IMMUNE CELLS

Using peripheral blood mononuclear cells (PBMCs) from 15 healthy, 23 influenza-infected, and 22 CM/ID-19-infected subjects, the composition and activation of circulating leukocytes was examined with flow cytometry. Multivariate linear regression with subject age, sex, ethnicity, symptom duration at study enrollment, and all comorbidities as covariates were used to explore immune cell dynamics as a function of condition while statistically controlling for demographic and other clinical differences across the patient groups. Circulating immune cells were initially characterized by quantifying the absolute number of CD4⁺ and CD8⁺ T lymphocytes and CD19⁺ B cells. COVID-19 and influenza subjects exhibited trends of decreased B cells and significant reductions in both T cell subsets, which generally constitute most of the circulating PBMCs in healthy controls. In contrast, COVID-19 subjects had significantly more circulating early antibody-secreting B cell plasmablasts than controls. Circulating activated CD4⁺ and CD8⁺ cells were equivalent across all groups. However, when compared with either influenza or control subjects, COVID-19 subjects exhibited significantly reduced numbers of circulating monocytes, including all three common classifications of human monocytes (classical, intermediate, and nonclassical).

Given the pronounced variation in monocyte abundance across patient conditions, major histocompatibility complex class II expression on the surface of monocytes was also measured to gauge monocyte activation. It was noted that COVID-19 subjects had reduced abundances of HLA-DR on the surface of all classes of monocyte when compared with influenza subjects or controls, although only intermediate monocytes reached statistical significance after controlling for covariate effects. In addition, patients with COVID-19 exhibited significantly less surface HLA-DR on CD8⁺ T cells than patients with influenza and trends toward less HLA-DR on CD4⁺ T cells in comparison to both patients with influenza and healthy controls. Once again using multivariate linear regression, potential differences in HLA-DR abundance between patients with moderate illness and those with severe illness was assessed, wherein severe illness was defined as those who required intubation and mechanical ventilation or who ultimately expired as a result of their illness. Although there were no associations with severity in HLA-DR abundance among lymphocyte populations, it was found that, compared to moderately ill patients, the severest patients exhibited substantially less HLA-DR on intermediate and nonclassical monocytes.

### EXAMPLE 4: EVALUATION OF CYTOKINE ASSOCIATIONS WITH DISEASE

From the primary cohort, plasma cytokine levels were measured from 79 patients with SARS-CoV-2 (COVID-19) infection, 26 patients with confirmed influenza virus infection, and 8 healthy controls. Among the patients with COVID-19, two response profiles were immediately apparent, with 3 of 79 patient samples exhibiting obviously distinct cytokine profiles in principal components analysis (PCA). These samples were characterized by cytokine levels of >2 SDs from the mean for more than 17 of the 35 cytokines measured (range: 49 to 89%), encompassing broad and unfocused immune responses characteristic of classic cytokine storm. Cytokine storms in other conditions have been defined by extreme deviations in the levels of a broad array of cytokines rather than just moderate elevations in targeted pathways (Guo & Thomas (2017) Semin. Immunopathol. 39:541-550). Standard deviations (SDs) from the mean ranged from 2 to 10.5 among these cytokine storm syndrome (CSS) subjects, with outlier values ranging from 0.8 to 2 orders of magnitude higher than the mean for each of the measured cytokines. Patients with CSS were all African American, one female (89 years) with no noted comorbidities, one female (62 years) with diabetes mellitus, and a male (47 years) with diabetes mellitus and preexisting chronic pulmonary disease. All three patients with CSS were admitted to the ICU, required intubation and mechanical ventilation, and ultimately expired. In subsequent analyses, these three patients were visualized and analyzed as a separate CSS group unless otherwise noted. Among the remaining 76 COVID-19 samples from this primary cohort, patients often exhibited values outside 1.5 interquartile ranges for individual cytokines, suggestive of activation of specific pathways; however, the vast majority of each patient's individual cytokine levels were well within the majority of the observed variation, which does not support the broad dysregulation of cytokines expected in a CSS phenotype.

It has been previously shown that many cytokines are often correlated with demographic and environmental factors (e.g., age and prior herpesvirus exposure) (Ostansky, et al. (2014) Am. J. Respir. Crit. Care Med. 189:449-462). To assess cytokine differences across groups while controlling for these potentially confounding factors, including the significant differences in age observed within the cohort, estimated marginal means from linear regression models were generated that incorporated age, sex, ethnicity, days since symptom onset at enrollment, and all reported comorbidities as covariates. In comparison to healthy controls, both influenza and COVID-19 subjects exhibited elevated levels of a number of cytokines. Among patients with COVID-19, IP-10, IL-8, MCP1, HGF (hepatocyte growth factor) and MIP-1β were significantly up-regulated compared to healthy controls, in addition to apparent (but not statistically significant) trends for increases in MIG (monokine induced by interferon gamma), granulocyte-macrophage CSF (GM-CSF), IL-1RA, IL-2, IL-17f, and IL-6. In comparison to healthy controls, influenza-infected patients likewise exhibited significant up-regulation of all of cytokines up-regulated among patients with COVID-19, but influenza-infected patients also exhibited significantly greater abundances (compared to COVID-19-infected patients) of a number of cytokines with known inflammatory and immunomodulatory roles, including MIG, IL-1RA, IL-2R, IL-2, IL-17f, and IL-12.

To elucidate the unique ways in which COVID-19 modulates cytokine profiles, the majority of cytokine analyses was focused on comparisons between the COVID-19 and influenza groups. Direct comparisons between infected groups revealed that the dominant response profile among patients with COVID-19 consisted of more selective cytokine up-regulation, with a relative bias toward lower inflammation when compared to patients with influenza. Among all subjects, it was found that for 28 of 35 cytokines, patients with COVID-19 had lower median cytokine levels, although not all were statistically significant. Among the statistically significant reduced cytokines exhibited by patients with COVID-19 compared to influenza patients were interferon-γ (IFN-γ), MIG, IL-1RA, IL-2R, G-CSF, IL-17a, IL-9, and MIP-1α. Upon visual inspection of the raw data, both IL-6 and IL-8 appeared to be greater among COVID-19 than influenza-infected patients, but the estimated marginal means of neither cytokine was significantly different after controlling for covariates, particularly the effects of age, sex, and preexisting pulmonary disease. This finding is particularly relevant in the ongoing discussion of COVID-19 cytokine profiles: Although some cytokines seem to be higher among patients with COVID-19, those differences are potentially inflated by other underlying demographic and clinical differences. For instance, hospitalized patients with COVID-19 are generally older (Table 1; Richardson, et al. (2020) JAMA 323:2052-2059), and many baseline cytokine levels increase with age (22, 23). When accounting for these and other confounding factors, the data indicated that most patients with COVID-19 did not have an inflammatory phenotype as profound as patients with influenza, with certain targeted exceptions.

### EXAMPLE 5: VALIDATION OF COVID-19 CYTOKINE PATTERNS IN AN ADDITIONAL SARS-CoV-2 COHORT

To validate the cytokine patterns observed among COVID-19, influenza, and healthy control groups in the primary cohort, a follow-up cohort was enrolled that was composed of the next 89 consecutive confirmed patients with COVID-19 enrolled into the ongoing prospective observational study (Tables 3 and 4).

TABLE 3

| | SARS- | Validation- | Validation- | Validation- |
|---|---|---|---|---|
| | COV-2 | Primary COVID- | Healthy | Influenza |
| | (n = 89) | 19 Comparison | Comparison | Comparison |
| Demographics | | | | |
| Means ± SD | 61 ± 17 | P = 1, | P < 0.001, | P = 0.041, |
| (range) | (19-92) | N.S. | OR = 0.87 | OR = 0.93 |
| age, in | | | | |
| years | | | | |
| Female | 39% | P = 1, | P = 1, | P = 1, |
| | 35/89) | N.S. | N.S. | N.S. |
| Ethnicity | | | | |
| African | 74% | — | — | — |
| American | (66/89) | | | |
| White | 25% | P = 1, | P = 0.069, | P = 1, N.S. |
| | (22/89) | N.S. | N.S. | |
| Other | 1% (1/89) | P = 1, N.S. | — | P = 1, N.S. |

N.S., not significant.

TABLE 4

| | | Validation- | Validation- |
|---|---|---|---|
| | SARS- | Primary | Influenza |
| | COV-2 | COVID-19 | Comparison |
| | (n = 89) | Comparison | n |
| Clinical Characteristics | | | |
| Mean (IQR) body mass | 28.6 | — | — |
| index | (24-33) | | |
| Mean (IQR) symptom | 7.5 | P = 0.958, | P = 0.635, |
| duration at study | (2-9) | N.S. | N.S. |
| enrollment, in days | | | |
| Hospital admission | 94% | P = 0.958, | P = 0.043, |
| | (84/89) | N.S. | OR = 0.046 |
| ICU admission | 48% | P = 0.10, | P = 1, |
| | (43/89) | N.S. | N.S. |
| Intubation and | 27% | P < 0.001, | P = 0.893, |
| mechanical ventilation | (24/89) | OR = 19.03 | N.S. |
| In-hospital death | 17% | P = 0.509, | P = 1, |
| | (15/89) | N.S. | N.S. |
| Comorbidities | | | |
| Immunocompromised | 8% | P = 1, | P = 0.347, |
| | (7/89) | N.S. | N.S. |
| Chronic lung disease | 16% | P = 0.017, | P = 0.049, |
| | (14/89) | OR = 5.9 | OR = 11.69 |
| Chronic heart failure | 15% | P = 1, | P = 0.817, |
| | (13/89) | N.S. | N.S. |
| End-stage renal failure | 2% | P = 1, | P = 1, |
| | (2/89) | N.S. | N.S. |
| Diabetes mellitus | 44% | P = 1, | P = 1, |
| | (39/89) | N.S. | N.S. |
| Active cancer | 3% | P = 0.893, | P = 0.387, |
| | (3/89) | N.S. | N.S. |

N.S., not significant.

Cytokine data from these patients were collected and analyzed as described for the primary cohort. Among the 89 patients in the validation cohort, four exhibited marked variation in their cytokine profiles that, although not as extreme as those in the primary cohort, were consistent with a CSS phenotype. These samples were characterized by cytokine levels of >2 SDs from the mean for more than 9 of the 35 cytokines measured (range: 26 to 49%). Two of these patients self-identified as African American (59-year-old female, 71-year-old male), one as “other” (41-year-old male), and one as white (64-year-old male). Both African-American patients with CSS had been previously diagnosed with diabetes mellitus, and the 59-year-old female also had been diagnosed with chronic heart failure and chronic pulmonary disease and was immunosuppressed. The other patients with CSS had no listed comorbidities. The 64-year-old male patient was admitted to the ICU, required intubation and mechanical ventilation, and ultimately expired. None of the other three patients with CSS were intubated, and all ultimately survived. In contrast to these CSS profiles, another subset of three patients exhibited a unique profile with a classic T_{H}22 signature, including of production of high levels of IL-22, GM-CSF, and IL-13 without high levels of IL-17. All patients with a T_{H}22 signature were female African Americans (aged 19, 26, and 71) with no listed comorbidities. None required intubation, and all survived. Although samples exhibiting the T_{H}22 signature are plotted separately for visual comparison, they were considered to represent regulated cytokine responses and were therefore included as COVID-19 validation samples in all statistical analyses.

Comparison of cytokine measures from the validation cohort to the original healthy controls and patients with influenza were largely consistent with those observed in the primary cohort. In addition to significantly higher levels of IP-10, IL-8, HGF, and MIP-1β observed among patients with COVID-19 from the primary cohort in comparison to healthy controls, the validation cohort also exhibited significantly greater levels of IL-12, epidermal growth factor (EGF), and IL-2, two of which were consistent with trends observed in the primary cohort analyses.

In comparison to influenza-infected subjects, the COVID-19 subjects in the validation cohort also exhibited many of the same patterns observed within the primary COVID-19 cohort with the single exception of MIP-1α (P=0.14), which may have differed due to variations in the assay's lower limits of detection. In addition, the validation COVID-19 cohort in comparison to influenza-infected subjects exhibited significantly lower levels of IL-1β, IL-4, IP-10, TNFα, IL-1α, IL-17f, fibroblast growth factor (FGF), and eotaxin, several of which were consistent with nonsignificant trends observed in the primary cohort analyses. Again, no significant difference in either IL-6 or IL-8 between influenza-infected and COVID-19-infected patients was detected after controlling for demographic and clinical covariates. Overall, these data validated the observation in the primary cohort that many cytokines are down-regulated in patients with COVID-19 compared to influenza-infected patients, indicating that overall higher inflammation is foremost predictive of influenza infection and that a defining feature of COVID-19 disease is generally reduced inflammation compared to influenza; aside from the 4% of patients with extreme cytokine dysregulation (i.e., CSS), COVID-19 subjects were not characterized by overall high levels of cytokines but rather exhibited a selective pattern of inflammation in which only a subset of inflammatory cytokines were up-regulated, and most were down-regulated when compared with seasonal influenza patients.

### EXAMPLE 6: CROSS-COHORT COMPARISONS AND INTEGRATED CYTOKINE ANALYSES

Comparison of the validation cohort to the COVID-19 group from the primary cohort revealed no significant differences in demographics or comorbidities, save for a significant reduction in preexisting chronic lung disease (16% in the validation cohort compared to 34% in the original cohort; Table 4). Of the other clinical characteristics considered, the validation cohort was significantly less likely to receive mechanical ventilation (27% compared to 44%). It was hypothesized that this difference reflects the evolution of treatment approaches over time rather than an underlying difference in the patient population, as the difference in ventilation rates was significant after controlling for differences in comorbidities, and there was no significant difference in death rates between the cohorts. Furthermore, written guidance to the clinical staff at the study institutions over the course of the first weeks of the pandemic reflected a nationwide transition from initial recommendations for early and aggressive intubation of patients with COVID-19 with relatively moderate hypoxia to later written guidance that focused on maximizing oxygen delivery strategies before intubation using more traditional approaches to acute respiratory failure. Although a significant negative association between the week of enrollment and the percentage of patients intubated was found, the opposite relationship between the week of enrollment and the percentage of patients who survived infection was observed. These correlations indicate that aggressive early intubation strategies may have been unwarranted or even harmful. An alternative hypothesis is that disease severity somehow naturally declined over time among patients with COVID-19 seeking hospital care in the St. Louis area.

Regardless of these differences, many of the underlying cytokine patterns were replicated between the primary and validation cohorts, as most of the cytokine variation among the two COVID-19 cohorts overlapped substantially. Yet, other patterns seemed not to replicate to statistical significance simply due to a lack of power for the highly parameterized models required to control for all demographic and clinical factors (Oshansky, et al. (2014) Am. J. Respir. Crit. Care Med. 189:449-462). This was not particularly unexpected as the relatively high dimensionality of cytokine data and the tendency for extreme outliers can make it difficult to detect statistically significant associations while conservatively controlling for false discovery rate. To address this, the cytokine data from the two COVID-19 cohorts was integrated by using a data-driven modular informatics approach developed specifically for cytokine analyses (Cohen, et al. (2019) Front. Immunol. 10:1338). These analyses allowed for the detection of a number of co-correlating cytokines across COVID-19 samples, which were grouped into distinct coexpression modules using hierarchical clustering. This unsupervised approach categorized the 35 cytokines assayed among COVID-19 samples into eight distinct modules of cosignaling cytokines, including a module composed of HGF, IL-1RA, IL-6, and IL-8 (module 1). As described above, most elements of this module were independently found to be up-regulated in both influenza and COVID-19 groups compared to healthy controls and were comparably expressed between COVID-19 and influenza groups, save for IL-1RA, which showed decreased expression among COVID-19 subjects compared to influenza subjects. Plasma IL-6 and IL-8 have been shown to correlate strongly in large pediatric and adult influenza cohorts, albeit typically with G-CSF and MIP-1α as well (Cohen, et al. (2019) Front. Immunol. 10:1338); in contrast, among COVID-19 samples, G-CSF clustered with IL-1α, IL-9, and TNFα (module 3), whereas MIP-1α clustered with 13 other cytokines, including several ILs and IFN-α (module 5). Module 5 contained cytokines associated with type 1 (IL-12), type 2 (IL-4), and type 3 (IL-17f) immune responses, including those secreted by innate (IFN-α and IL-1) and adaptive (IL-4 and IL-15) immune cells. Notably, several cytokines were assigned to their own modules due to a lack of sufficient correlation with others, including the chemokine RANTES (module 8), vascular endothelial growth factor (VEGF; module 2), and IFN-γ (module 7). In general, these analyses suggest that aspects of typical cytokine cosignaling modules inferred from large cohorts in other severe respiratory diseases are altered in the context of COVID-19, indicative of a unique immunoregulatory environment in COVID-19 potentially demonstrated by the overall reduced inflammatory profile. The significantly lower levels of IFN-γ and its lack of clustering with other cytokines indicates that this prototypical type 1 cytokine is not being produced in a manner typical of other common viral infections.

Using this analytical framework further, it was next determined whether particular cosignaling modules or individual cytokine expression patterns were associated with clinical outcomes among patients with COVID-19. Here, multivariate logistic regression was used, again controlling for age, sex, ethnicity, days since symptom onset at enrollment, and all reported comorbidities to calculate adjusted odds ratios of severity. Because these analyses spanned both primary and validation cohorts, cohort was also included as a covariate to control for any potential cryptic differences between the patient populations, and multiple comparisons (13 covariates tested within each model) were adjusted for by controlling the false discovery rate. These analyses revealed a number of significant associations between various measures of disease severity, cosignaling cytokine modules, and their constituent cytokines. For instance, increases in cytokine modules 1, 2, and 6 were associated with increased odds of requiring ICU admission, as were HGF, IL-1RA, IL-6, IL-8, VEGF, G-CSF, IL-15, IL-1β, MCP1, MIP-1α, and MIG individually. Increases in module 1 and most of its constituent cytokines, as well as G-CSF and IL-1β, were likewise associated with increased risk of death. While some of these cytokines (IL-6 and IL-8) have been implicated previously in COVID-19 pathogenesis, the comprehensive screening approach herein has identified a signature that is notable for its focus on largely innate inflammatory mediators associated with monocyte and neutrophil mobilization. In addition, in this analysis, there was a lack of association with traditional adaptive inflammatory markers (IFN-γ, IL-13, and IL-5) that has been observed in some other reports (Lucas, et al. (2020) Nature 584:463-9). Strikingly, this severity signature is reminiscent of the innate monocyte mobilization signature identified in patients with influenza using a similar statistical approach, with the notable lack of IFN-α associations (Oshansky, et al. (2014) Am. J. Respir. Crit. Care Med. 189:449-462; Cohen, et al. (2019) Front. Immunol. 10:1338).

### EXAMPLE 7: SINGLE-CELL TRANSCRIPTIONAL PROFILES OF COVID-19 SUBJECTS WITH RESPIRATORY FAILURE ARE CONCORDANT WITH SIGNALS OF TARGETED IMMUNOSUPPRESSION

Immune suppression can often occur as a negative feedback from immune activation. Thus, further resolution of the immune state of a subset of severe COVID-19 subjects was sought to understand the dominant regulatory signals determining their trajectory. A total of 37,469 cells from eight subjects (three COVID-19-infected, three influenza-infected, and two healthy controls) were obtained for single-cell gene expression analyses after standard processing and filtering. All six of the infected subjects required intubation and mechanical ventilation for severe respiratory failure, and ultimately three of the COVID-19-infected patients and one of the three influenza-infected patients died from their illnesses. Using an integration-based approach that leverages convergent expression signals across samples (see Example 1), 25 putative transcriptional clusters were identified that were categorized into major cell subsets, including monocytes and macrophages (four transcriptional clusters including CD16⁺ and CD16⁻ monocytes), CD8⁺ T cells (three clusters including putative naive, effector, and central memory populations), CD4⁺ T cells (two clusters including putative naive and memory populations), regulatory T cells (T_{regs}), innate-like T cells (including gamma delta T cells and MAIT cells (mucosal-associated invariant T cells)), B cells (two clusters including putative memory B cells), plasmablasts, mixed cytolytic lymphocyte populations ((MCLPs) four clusters that potentially include natural killer (NK) and NKT cells), platelets, red blood cells, granulocytes (putative), stromal cells, and plasmacytoid dendritic cells (PDCs), as well as putative doublets.

As variations in the relative abundance of subsets across groups were obvious upon visual inspection, each of these major groups and their constituent transcriptional clusters were interrogated for variation in both relative abundance and gene expression owed to differences in group (i.e., COVID-19-infected, influenza-infected, or healthy control). Although some transcriptional clusters had more cells from one condition or another, an analytical approach was used that allowed for detecting differences between conditions by simultaneously assessing the proportions of cells expressing a gene and the expression of that gene within cells expressing it.

Given the substantial down-regulation of HLA-DR among COVID-19 monocytes observed during flow cytometry analysis, potential transcriptional differences between COVID-19 and influenza specifically within the transcriptionally defined monocyte/macrophage subset and clusters were further investigated. As expected, given the flow cytometry analysis carried out on these same patients, the proportion of cells in monocyte/macrophage clusters were substantially smaller in COVID-19 compared to both healthy and influenza subjects. In addition, expression of the conserved class II chain HLA-DR was significantly reduced in cells from patients with COVID-19 compared to patients with influenza. At the single-cell transcriptional level, many of the genes that were profiled in the protein cytokine assays were rarely observed, likely due to the sensitivity limitations of the gene expression kit. Therefore, Gene Set Enrichment Analysis (GSEA; Subramanian, et al. (2005) Proc. Natl. Acad. Sci. USA 102:15545-50) was used to broadly survey transcriptional variation as a function of infection status in an unbiased manner. Specifically, gene expression differences between COVID-19-infected and influenza-infected patients were ranked for each subset and enrichment of Hallmark gene sets as a function of these diagnoses were tested. Unexpectedly, a number of important immunological pathways were significantly enriched, specifically among cells from patients with influenza across a number of subsets: Compared to the influenza condition, both IFN-γ and IFN-α response pathways were significantly down-regulated within the COVID-19 condition for B cells, plasmablasts, CD8⁺ T cells, MCLPs, T_{regs}, PDCs, and monocyte/macrophage subsets. These pathway-based analyses were particularly informative, as there was no significant difference in expression of IFN-γ itself in these subsets, and IFN-α transcripts were not detected at all. More exhaustive analysis using gene ontology pathways related to interferon production, secretion, response, and regulation demonstrated that patterns observed among IFN-γ and IFN-α pathways extended to IFN-β (which was also not directly detected in the transcriptomic data) and general type I interferon pathways across most subsets but particularly among monocytes. These patterns were concordant with substantial enrichment of inflammatory pathways in influenza cells compared to COVID-19 cells across a majority of cell subsets. In contrast, COVID-19 cells were significantly enriched for a number of pathways involved in cellular metabolism, stress, corticosteroid stimulus, and proliferation in comparison to influenza cells across most subsets.

To confirm these observations, the specific expression of Stat genes was analyzed. These genes included the IFN-associated signal transducers and activators of transcription 1 (STAT1) and STAT2, which were both significantly underrepresented in patients with COVID-19 compared to patients with influenza. STAT3, which is critical for IL-6 signaling, was also expressed significantly less in patients with COVID-19 compared to patients with influenza despite the elevated levels of IL-6 circulating in these subjects. Together, these data indicate that, in this subset of patients with COVID-19, there was a general refractoriness to certain inflammatory signals, including IFNs. Although the transcriptional signals from this subset of patients were concordant with the robust phenotypic patterns observed across the much larger cohorts, it is important to note that others have reported dissimilar findings in their own transcriptional comparisons of treated influenza-infected and COVID-19-infected patients (Lee, et al. (2020) Sci. Immunol. 5:eabd1554).

Excessive glucocorticoid production appears to account for the observed immune dysregulation and disease manifestations in COVID-19. In particular, the relatively high levels of IL-6 production can directly drive excessive cortisol production through multiple mechanisms, including through the direct induction of corticotropin-releasing hormone and adrenocorticotropin. IL-6 can also act directly on the adrenal cortex to stimulate GC release (Weber, et al. (1997) Endrocrinology 138:2207-10). While GCs are generally immunosuppressive, which is why they are often used therapeutically, their effects are uneven across the cytokine landscape, with cortisol failing to suppress IL-6 (DeRijk, et al. (1997) J. Clin. Endocrinol. Metab. 82:2182-91) or even inducing IL-6 and IL-8 in one report (DaPozzo, et al. (2018) PLoS ONE 13:e0200924). In another retrospective cohort study, increased cortisol levels were observed in a majority of patients with COVID-19 (Schroeder et al. (2020) MedRxiv 2020.05.07.20073817), while another analysis found an association between risk of COVID-19 mortality and high levels of serum cortisol (Tan, et al. (2020) Lancet Diabetes Endocrinoi. 8:659-660).

In contrast, a report from the RECOVERY trial has suggested that treatment with steroids (dexamethasone) had a significant protective effect in reducing mortality among the most severe COVID-19 patients (The RECOVERY Collaborative Group (2020) N. Engl. J. Med. 10.1056/NEJMoa2021436). These data demonstrated a statistically significant reduction in mortality among patients requiring oxygen (21 versus 25%) or ventilation (29 versus 40%). In light of the findings presented herein, it is posited that this subset of patients that was aided (˜5 to 11% of the most severe patients and approximately 2% of the total cohort of COVID-19 patients enrolled in the study) represented those who had the severe cytokine storm immunotype. Intriguingly, in those patients not receiving respiratory support, outcomes for patients on dexamethasone were poorer, with 17% mortality versus 13.2% in the control group (although this was not statistically significant; P=0.14). Other reports have similarly found deleterious effects of GC administration in COVID-19 and related illnesses (Russell, et al. (2020) Lancet 395:473-5). On the basis of the results herein, this lack of efficacy in the larger population may correspond to patients already experiencing higher levels of GC production. Thus, therapeutic consideration should be given to inhibiting both IL-6 and GC activity in most of the patients with COVID-19 exhibiting this phenotype (high IL-6, low IFN signaling, and profound cytopenias) versus the small proportion of patients with a true cytokine storm phenotype.

1. A method for treating a viral infection comprising administering to a subject with a viral infection an effective amount of a cortisol antagonist that inhibits cortisol receptor binding or inhibits cortisol synthesis thereby treating the subject's viral infection.

2. The method of claim 1, wherein the viral infection is a coronavirus infection.

3. The method of claim 2, wherein the coronavirus infection is SARS-CoV-2.

4. The method of claim 1, further comprising administering an IL-6 antagonist.

5. A method for reducing the severity of a viral infection comprising administering to a subject with a viral infection an effective amount of a cortisol antagonist that inhibits cortisol receptor binding or inhibits cortisol synthesis thereby decreasing the severity of the subject's viral infection.

6. The method of claim 5, wherein the viral infection is a coronavirus infection.

7. The method of claim 6, wherein the coronavirus infection is SARS-CoV-2.

8. The method of claim 5, further comprising administering an IL-6 antagonist.

9. A kit comprising (a) a cortisol antagonist that inhibits cortisol receptor binding or inhibits cortisol synthesis and (b) an IL-6 antagonist.

10. A method for treating a viral infection comprising administering to a subject with a viral infection an effective amount of a pharmaceutical composition consisting essentially of a cortisol antagonist and optionally IL-6 thereby treating the subject's viral infection.

11. The method of claim 10, wherein the viral infection is a coronavirus infection.

12. The method of claim 11, wherein the coronavirus infection is SARS-CoV-2.

13. A method for reducing the severity of a viral infection comprising administering to a subject with a viral infection an effective amount of a pharmaceutical composition consisting essentially of a cortisol antagonist and optionally IL-6 thereby decreasing the severity of the subject's viral infection.

14. The method of claim 13, wherein the viral infection is a coronavirus infection.

15. The method of claim 14, wherein the coronavirus infection is SARS-CoV-2.
